(19) United States
(12) Patent Application Publication
(10) Pub. No.: US 2015/0321005 A1
(43) Pub. Date: Nov. 12, 2015

(54) HEART STIMULATOR FOR IMPLANTATION IN A HEART VENTRICLE

(71) Applicant: Biotronik SE & Co., KG, Berlin (DE)

(72) Inventors: Thomas DOERR, Berlin (DE); Michael DIEBOLD, Berlin (DE); Nicolas DEGEN, Beringen (CH)

(73) Assignee: BIOTRONIK SE & CO. KG, Berlin (DE)

(21) Appl. No.: 14/802,991

(22) Filed: Jul. 17, 2015

Related U.S. Application Data
(60) Provisional application No. 61/038,849, filed on Mar. 24, 2008.

(51) Int. Cl. A61N 1/368; A61N 1/365; A61N 1/375; A61N 1/05

(52) U.S. Cl. CPC A61N 1/3686; A61N 1/057; A61N 1/36521; A61N 1/3756

(57) ABSTRACT

Embodiments include a heart stimulator that may be implanted in a heart ventricle and that includes a housing. The housing includes fixation elements that passively fix the heart stimulator in a heart ventricle and electrode poles that one or more of deliver stimulation pulses and sense electrical potentials. The housing includes an energy supply unit, a control unit connected to the energy supply unit, and a stimulation pulse generator connected to the control unit and the energy supply unit. A plurality of electrode poles are distributed on a surface of the housing and a switching matrix is connected between the electrode poles and the stimulation pulse generator, wherein the stimulation pulse generator is electrically connected via the switching matrix to different electrode poles depending on a switched state of the switching matrix.

### BACKGROUND OF THE INVENTION

1. Field of the Invention

Embodiments of the invention generally relate to heart stimulators that may be implanted in a heart ventricle. In embodiments, the heart stimulators, such as leadless pacemakers, include a housing, which includes fixation elements to actively or passively fix the heart stimulator in a heart ventricle, and electrode poles to one or more of deliver stimulation pulses and sense electrical potentials. In embodiments, the heart stimulators include an energy supply unit, such as a battery or a generator, a control unit connected to the energy supply unit, and a stimulation pulse generator connected to the control unit and the energy supply unit.

2. Description of the Related Art

Generally, heart stimulators, such as leadless pacemakers, which may be implanted in a heart ventricle, are known in principle. The advantage of a heart stimulator that may be implanted directly in a heart ventricle is that, typically, a heart stimulator of this type does not require any electrode lines leading from a stimulation impulse generator in the housing of the heart stimulator to stimulation electrode poles in the respective heart ventricle. Rather, generally, a heart stimulator of this type may be arranged directly in the heart ventricle, such that electrode poles, which serve to deliver stimulation pulses, may be fitted directly on the housing of the heart stimulator.

Generally, heart stimulators that may be implanted in a heart ventricle may be anchored in the heart ventricle in various ways. By way of example, generally, heart stimulators with fixation elements for the active fixing of the heart stimulator are known. Fixation elements of this type, typically, have to be actuated in order to be anchored in a respective heart ventricle. An advantage of this active fixing is that, generally, a defined position and orientation of the heart stimulator in the respective heart ventricle may typically be attained with the active fixing.

Typically, heart stimulators with fixation elements for passive fixing do not have to be actively fixed and may therefore be implanted more easily. However, generally, the disadvantage that heart stimulators of this type is that they also have to be oriented during the implantation such that a respective stimulation electrode pole, for example a tip electrode, rests against the myocardium of the respective heart ventricle, such that a respective stimulation impulse may act effectively. As such, generally, the stimulation stimulus threshold (and thus the energy required for a respective effective stimulation of the myocardium) is as low as possible. Typically, an orientation of this type of the heart stimulator is not always very easy and presupposes that the trabecular structures of the heart surrounding the heart stimulator, the fixation elements and the stimulation electrode pole are disposed relative to one another in a suitable geometric arrangement. In addition, generally, there is the problem that passively fixable heart stimulators in particular may move slightly after implantation. Generally, microdislocations may occur, which may have an influence on the stimulus threshold.

In view of the above disadvantages of passively fixable heart stimulators, there is a need of effectively functioning, passively fixable heart stimulators to be implanted in a heart ventricle.

### BRIEF SUMMARY OF THE INVENTION

One or more embodiments of the invention provide a heart stimulator that may be implanted in a heart ventricle. In at least one embodiment, the heart stimulator includes a housing that may include fixation elements to passively fix the heart stimulator in a heart ventricle, and a plurality of electrode poles to one or more of deliver stimulation pulses and sense electrical potentials. In at least one embodiment, the housing may include an energy supply unit, a control unit connected to the energy supply unit, and a stimulation pulse generator connected to the control unit and the energy supply unit. One or more embodiments may include a plurality of the electrode poles distributed on a surface of the housing, and a switching matrix connected between the electrode poles and the stimulation pulse generator. As such, in at least one embodiment, the stimulation pulse generator may be electrically connected via the switching matrix to different electrode poles depending on a switched state of the switching matrix.

In one or more embodiments, the electrode pole of the heart stimulator at which the electric field is concentrated in the event of a stimulation pulse delivery, such as a cathode, also referred to herein as the stimulation electrode pole, may not be determined from the outset. Rather, in at least one embodiment, one of a plurality of electrode poles arranged on the housing of the heart stimulator may be connected via the switching matrix to the stimulation pulse generator, such that one of the electrode poles serves as a stimulation electrode pole, whereas for example the other electrode poles or at least one of the remaining electrode poles serve as a respective opposite pole.

One or more embodiments include a miniaturized heart stimulator, which may be implanted very easily and reliably in the trabecular meshwork of a heart ventricle, for example a right ventricle of a human heart.

In at least one embodiment of the invention, the number of electrode poles arranged on the housing of the heart stimulator may include at least three or at least four, and wherein each pair of the electrode poles on the surface of the housing may include, at least an approximately identical distance from one another. One or more embodiments may include a distribution of the electrode poles that is uniform, or as uniform as possible, over the surface of the housing of the heart stimulator, such that the orientation of the electrode poles is no longer decisive in the event of implantation of the heart stimulator.

By way of at least one embodiment, the heart stimulator may include a sensing unit, which is connected via the switching matrix to the electrode poles and which may sense electrical values for each of the electrode poles or pairs of electrode poles. In one or more embodiments, the electric values may allow a stimulation success prognosis for a respective electrode pole or a respective pair of electrode poles. In at least one embodiment, the sensing unit may be connected to the control unit, and the control unit may select the electrode pole or the pair of electrode poles that promises, or indicates, a greatest stimulation success. In one or more embodiments, such a heart stimulator may automatically select, as a stimulation electrode pole, the electrode pole or pair of electrode poles of which the stimulation stimulus threshold is lowest. For example, at least one embodiment of the invention may include a right-ventricular passively fixable leadless pacemaker with a multiplicity of electrode poles and an automatic selection of the stimulation electrode poles.

In one or more embodiments, the switching matrix may be connected to the control unit, and the control unit may electrically connect the electrode pole or the pair of electrode poles that promises, or indicates, a greatest stimulation success to the stimulation pulse generator, at least for the duration of, or during, the delivery of a stimulation pulse. As such, in at least one embodiment, the control unit may determine not only the electrode pole for which a lowest stimulus threshold is provided, but may additionally ensure, via the switching matrix, that the selected electrode pole is also actually connected as a stimulation electrode pole to the stimulation pulse generator when the stimulation pulse generator generates and delivers a stimulation pulse.

In one or more embodiments, the control unit may shift the switching matrix, at least for the duration of the delivery of the stimulation pulse, into a switched state in which the electrode pole that promises or indicates a greatest stimulation success is electrically connected to the stimulation pulse generator as the electrode pole to be used as a cathode. As such, in at least one embodiment, the control unit may switch the switching matrix such that one of the electrode poles, which may serve as the stimulation electrode pole, is connected as the cathode.

In one or more embodiments, the remaining electrode poles may not serve as a stimulation electrode pole, and the control unit may shift the switching matrix, at least for the duration of the delivery of a stimulation pulse, into a switched state in which some or all of the remaining electrode poles are interconnected as an anode. In at least one embodiment, some or all of the remaining electrode poles are connected to the stimulation pulse generator such that an optimum current density is produced for the stimulation electrode pole connected as the cathode. As such, one or more embodiments provide a stimulation that is as effective as possible and is an advantage compared with conventional heart stimulators, which typically include just a single stimulation electrode pole and a single opposite pole. In typical heart stimulators, an automatic optimization of the field distribution is not possible.

In one or more embodiments, the control unit may create a stimulation success prognosis prior to each stimulation delivery or following each unsuccessful stimulation delivery. As such, in at least one embodiment, the stimulation success prognosis may occur either beat-to-beat, for example prior to each stimulation pulse delivery, or may occur only when the delivery of a stimulation pulse does not cause any stimulation of the myocardium. In one or more embodiments, if the stimulation success prognosis occurs only when the delivery of a stimulation pulse does not cause any stimulation of the myocardium, the heart stimulator may include a stimulation success control unit or device, or stimulation success controller, such as a capture control unit or device or a capture controller. In at least one embodiment, the stimulation success control unit or device may determine whether an evoked stimulus response occurs following the delivery of a stimulation pulse. In one or more embodiments, the evoked stimulus response may be a corresponding electrical activity of the myocardium and a contraction of the myocardium and therefore of the heart ventricle. In at least one embodiment, the stimulation success control device may be used to select one of a number of electrode poles of a heart stimulator implantable in the heart as suitable stimulation electrode poles, as an advantage of the invention provided herein.

In one or more embodiments, the control unit may perform a stimulation success prognosis on the basis of one or more of a measured amplitude of an R wave (R amplitude), a measured impedance, a measured stimulus threshold, and a measured stimulus response. In at least one embodiment, a course of the electrical potential in the myocardium that is identifiable in an electrocardiogram and that originates from a depolarization of the cells of the myocardium and accompanies a contraction of the myocardium is referred herein to as an R wave. In one or more embodiments, a contraction of the myocardium as the result of an effective stimulation pulse delivery may be sensed directly by impedance measurement, for example. In at least one embodiment, the electrical stimulus response of the myocardium may be evaluated further, for example the period of time between stimulation pulse delivery and sensing of an induced stimulation response may be determined and used for a stimulation success prognosis. In one or more embodiments, a shorter time period may be an indication of a lower stimulus threshold. In one or more embodiments, the electrode pole with which there is a shortest time interval between delivery of a stimulation pulse and sensing of an evoked stimulus response, in each case compared with the other electrode poles, may be the best-suited stimulation electrode pole.

In at least one embodiment of the invention, electrical values that allow a stimulation success prognosis for a respective electrode pole or a respective pair of electrode poles, for example the amplitude of an R wave in the electrocardiogram (R amplitude), may include a measured impedance between two electrode poles, a stimulus threshold sensed using a stimulus threshold test, such as an electrode pole stimulus threshold test, or an electrical signal representing an evoked stimulus response.

In one or more embodiments, the housing of the heart stimulator may be, at least approximately, rotationally symmetrical. In at least one embodiment, the orientation of the housing in the event of the implantation is not decisive, and therefore the heart stimulator may be easily implanted.

According to one or more embodiments, each of the electrode poles may have at least approximately the same dimensions and the same electrical properties.

In at least one embodiment, the ratio of greatest spatial dimension to smallest spatial dimension of the housing may be less than 2, or less than 1.5, or less than 1. In one or more embodiments, for example with the ratio of greatest spatial dimension to smallest spatial dimension being less than 1, the housing may be spherical,.

In at least one embodiment, the volume of the housing may be between 0.5 and 2 cm³, and the weight of the heart stimulator may be less than 5 gr or less than 2 gr.

By way of one or more embodiments, the fixation elements may be rigid or flexible and may passively fix the heart stimulator in the trabecular meshwork of a heart ventricle. In at least one embodiment, such fixation elements may include tines. In one or more embodiments, the electrode poles may be arranged on the fixation elements themselves, for example the fixation elements may also form the electrode poles. As such, in at least one embodiment, the fixation elements may be formed as metal wires and are may each be electrically connected to the simulation pulse generator via the switching matrix.

In at least one embodiment, the heart stimulator may include permanent or temporary fixation elements to actively fix the heart stimulator. In one or more embodiments, the permanent or temporary fixation element may include a thread, for example, wherein the thread may be sewn on at a puncture site and may be resorbable. As such, at least one embodiment may include an active fixing only during the first weeks following an implantation of the heart stimulator. During the first weeks following the implantation, by way of at least one embodiment, the passive fixation elements may be connected in a stable manner to the trabecular meshwork of the respective heart ventricle. In one or more embodiments with the thread, the housing of the heart stimulator may include a thread mount, for example an eyelet.

In one or more embodiments, the energy supply unit may be an energy source, for example a battery, and/or may be a device that recovers energy, such as a generator. In at least one embodiment, the energy supply unit may include a nuclear battery.

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows a heart stimulator implanted in a right ventricle of a human heart;

FIG. 2 shows the heart stimulator from FIG. 1 in an enlarged isolated illustration; and

FIG. 3 shows a schematic block diagram of a heart stimulator according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

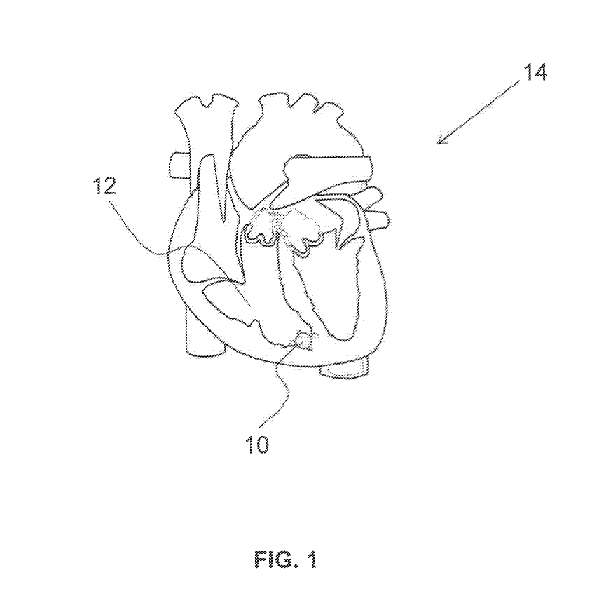

FIG. 1 shows a heart stimulator 10, such as a leadless pacemaker, which is arranged in the apex of a right ventricle 12 of a human heart 14, according to one or more embodiments of the invention. In at least one embodiment, the implantation site for a heart stimulator of this type includes the apex of the right ventricle 12. In one or more embodiments, the heart stimulator 10 may be fixed to the human heart 14 by “clamping” and “hooking” the heart stimulator 10 in the trabecular meshwork of the right ventricle 12. In one or more embodiments, due the configuration of the heart stimulator 10 (described in greater detail hereinafter), the orientation of the heart stimulator 10 does not play any role. As such, in at least one embodiment, the fixation elements of the heart stimulator 10 may be maximally effective.

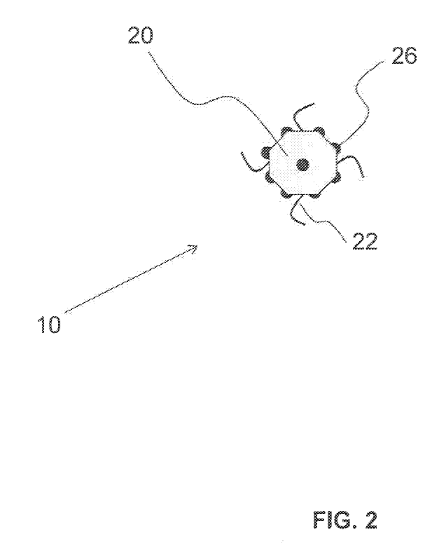

FIG. 2 shows the heart stimulator 10 in an enlarged isolated illustration, according to one or more embodiments of the invention. In at least one embodiment, the heart stimulator 10 includes a housing 20, which may include fixation elements 22 to passively fix the heart stimulator 10 in the trabecular meshwork of a heart ventricle. In one or more embodiments, the heart stimulator 10 may include a multiplicity or plurality of electrode poles 26 distributed on the surface of the housing 20.

In at least one embodiment, the housing 20 may be rotationally symmetrical, and the electrode poles 26 may be arranged on the surface of the housing 20 at equal distance from one another and distributed uniformly. In one or more embodiments, each of the electrode poles 26 may include an identical geometry and identical electrical properties.

In at least one embodiment, the passive fixation elements 22 may be formed as, or include, flexible barbs, and may be formed by, or include, electrically conductive metal wires. As such, in one or more embodiments, the fixation elements 22 may serve alternatively or additionally as electrode poles.

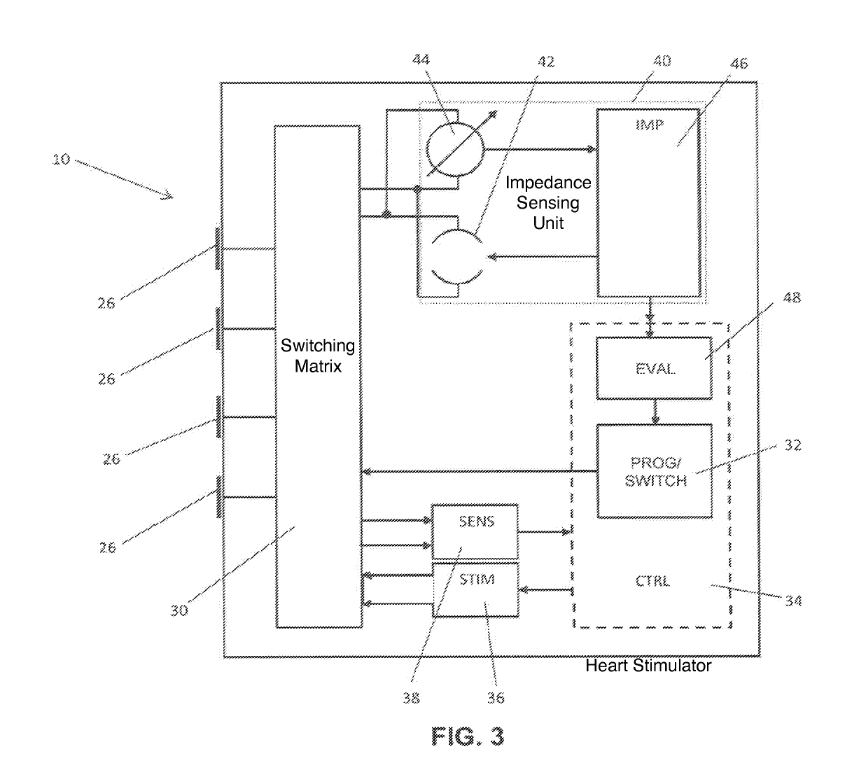

As discussed above, according to at least one embodiment of the invention, a great advantage of the heart stimulator 10 is that one of the plurality of electrode poles 26 may be selected automatically as the stimulation electrode pole and may be connected to a stimulation pulse generator to deliver the stimulation pulses. FIG. 3 shows a schematic block diagram of a heart stimulator according to one or more embodiments of the invention. FIG. 3 shows the components relevant to at least one embodiment of the invention. Further conventional components of a heart stimulator are not illustrated in the block diagram, for example, and may include one or more of an energy supply unit, a telemetry unit, an activity sensor, etc. by way of at least one embodiment.

The electrode poles 26 are illustrated schematically in FIG. 3, wherein the electrode poles 26 may be distributed uniformly over the periphery of the heart stimulator 10 in at least one embodiment of the invention. In one or more embodiments, the electrode poles 26 may all be connected to a switching matrix 30, which may be connected to a prognosis and switching unit 32 of a control unit 34. In at least one embodiment, the control unit 34 of the heart stimulator 10 may adjust the switched state of the switching matrix 30 via the prognosis and switching unit 32.

As shown in FIG. 3, by way of at least one embodiment, the switching matrix 30 may be connected to outputs and inputs of a stimulation pulse generator 36 and of a sensing unit 38 respectively. In one or more embodiments, the stimulation pulse generator 36 may generate stimulation pulses and may deliver the stimulation pulses via the switching matrix 30 and corresponding electrode poles 26 to surrounding tissue.

In at least one embodiment, the sensing unit 38 may be connected via the switching matrix 30 to individual electrode poles 26 or a plurality of the electrode poles 26, such that the sensing unit 38 may receive and sense and, where applicable, may evaluate, for example, electrical potential courses via the connected electrode poles in order to deliver corresponding signals to the control unit 34.

In one or more embodiments, the control unit 34 may evaluate the corresponding signals to trigger the generation and delivery of a stimulation pulse as necessary. In at least one embodiment, the control unit 34 may be connected to the stimulation pulse generator 36.

By way of one or more embodiments, the sensing unit 38 may be used to control stimulation success, such that the control unit 34 for example may perform an automatic stimulus threshold test, in which the control unit 34 allows the stimulation pulse generator 36 to deliver stimulation pulses of different intensity via a respective electrode pole connected as the stimulation electrode pole and via corresponding opposite electrodes. In at least one embodiment, following each stimulation pulse delivery, the sensing unit 38 may sense whether the stimulation was successful. In one or more embodiments, in order to effectively stimulate the myocardium, the intensity that a stimulation pulse delivered via a respective stimulation electrode pole must have may be determined. In at least one embodiment, the intensity is characteristic for the respective stimulus threshold, and may be different depending on which of the electrode poles 26 is connected as the stimulation electrode pole to the stimulation generator 36.

According to at least one embodiment, FIG. 3 shows an impedance sensing unit 40 including a current or voltage source 42, which may generate short, bi-phase subliminal pulses, wherein the subliminal pulses may be delivered via respective electrode poles to the myocardium. One or more embodiments may include a voltage or current measurement unit 44 that measures the voltage or current accompanying the respective delivery of a current or voltage pulse respectively. At least one embodiments of the invention may include an impedance determination unit 46, and an impedance evaluation unit 48 in the control unit 34. In one or more embodiments, the impedance determination unit 46 may be connected to the voltage or current measurement unit 44 to generate a characteristic variable for the respective impedance between the electrode poles and to deliver the characteristic variable to the impedance evaluation unit 48 in the control unit 34. As such, in one or more embodiments, the control unit 34 may sense a stimulation success, for example on the basis of impedance measurements, wherein the course of the impedance values for example may indicate a mechanical contraction of the respective heart ventricle.

According to at least one embodiment, the impedance sensing unit 40 may determine, using impedance measurement, the electrode poles that provide a good stimulation success prognosis. In one or more embodiments, the impedance sensing unit 40 may automatically select a stimulation electrode pole.

As discussed above, according to one or more embodiments, the control unit 34, following implantation of the heart stimulator 10, may perform the tests described herein by way of example in order to establish a stimulation success prognosis for each of the electrode poles 26. As such, in at least one embodiment, the control unit 34 may select the electrode pole 26 that provides or indicates the best stimulation success prognosis as the stimulation electrode pole and the cathode to deliver the stimulation pulse. Since changes in this regard may occur over the course of time, in at least one embodiment of the invention, the control unit 34 may perform the corresponding tests and therefrom establish a stimulation success prognosis immediately after the implantation of the heart stimulator 10, and may repeat the steps at later periods of time a number of times.

One or more embodiments of the invention include a very small leadless pacemaker, which may be implanted and passively fixed in a very simple and reliable manner, wherein, during the fixing, the position of the electrode poles may not be taken into consideration, since the heart stimulator may be introduced and hooked into the trabecular meshwork in any way.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

10 heart stimulator
12 ventricle
14 human heart
20 housing
22 fixation elements
26 electrode poles
30 switching matrix
32 prognosis and switching unit
34 control unit
36 stimulation pulse generator
38 sensing unit
40 impedance sensing unit
42 current or voltage source
44 voltage or current measurement unit
46 impedance determination unit
48 impedance evaluation unit

What is claimed is:

1. A heart stimulator configured to be implanted in a heart ventricle, comprising a housing, wherein the housing includes
a surface,
fixation elements that passively fix the heart stimulator in a heart ventricle,
a plurality of electrode poles that one or more of deliver stimulation pulses and sense electrical potentials,
wherein the plurality of electrode poles are distributed over the surface of the housing,
an energy supply unit,
a control unit connected to the energy supply unit,
a stimulation pulse generator connected to the control unit and the energy supply unit, and,
a switching matrix connected between the plurality of electrode poles and the stimulation pulse generator,
wherein the stimulation pulse generator is electrically connected via the switching matrix to different electrode poles of the plurality of electrode poles depending on a switched state of the switching matrix.

2. The heart stimulator as claimed in claim 1, wherein the plurality of electrode poles comprise at least 3 electrode poles or at least 4 electrode poles, and wherein each pair of the plurality of electrode poles on the surface of the housing comprise an at least approximately identical distance from one another.

3. The heart stimulator as claimed in claim 1, further comprising
a sensing unit connected via the switching matrix to the plurality of electrode poles,
wherein the sensing unit is configured to sense electrical values from each of the electrode poles or pairs of electrode poles of the plurality of electrode poles,
wherein said electrical values allow a stimulation success prognosis of a respective electrode pole or a respective pair of electrode poles of the plurality of electrode poles,
wherein the sensing unit is connected to the control unit, and,
wherein the control unit is configured to select an electrode pole or a pair of electrode poles from the plurality of electrode poles that indicates a greatest stimulation success.

4. The heart stimulator as claimed in claim 3, wherein the switching matrix is connected to the control unit, and wherein the control unit is configured to electrically connect the electrode pole or the pair of electrode poles of the plurality of electrode poles that indicates a greatest stimulation success to the stimulation pulse generator, at least for a duration of a delivery of a stimulation pulse.

5. The heart stimulator as claimed in claim 3, wherein the control unit is configured to shift the switching matrix at least for a duration of a delivery of a stimulation pulse into a switched state in which the electrode pole or the pair of electrode poles that indicates a greatest stimulation success is electrically connected to the stimulation pulse generator as the electrode pole or the pair of electrode poles to be used as a cathode.

6. The heart stimulator as claimed in claim 5, wherein the control unit is configured to shift the switching matrix at least for a duration of a delivery of a stimulation pulse into a switched state in which some or all other electrode poles of the plurality of electrode poles are interconnected as an anode and are connected to the stimulation pulse generator such that an optimum current density is produced for the electrode pole or the pair of electrode poles serving as the cathode.

7. The heart stimulator as claimed in claim 3, wherein the control unit is configured to perform a stimulation success prognosis prior to each stimulation pulse delivery or after each unsuccessful stimulation pulse delivery.

8. The heart stimulator as claimed in claim 3, wherein the control unit is configured to perform a stimulation success prognosis based on one or more of a measured R-wave amplitude, a measured impedance, a measured stimulus threshold, and a measured stimulus response.

9. The heart stimulator as claimed in claim 1, wherein the housing is at least approximately rotationally symmetrical.

10. The heart stimulator as claimed in claim 1, wherein each of the plurality of electrode poles comprises at least approximately identical dimensions and electrical properties.

11. The heart stimulator as claimed in claim 1, wherein the fixation elements form or comprise the plurality of electrode poles.

12. The heart stimulator as claimed in claim 1, wherein the housing further comprises a ratio of greatest spatial dimension to smallest spatial dimension of less than 2 or less than 1.5.

13. The heart stimulator as claimed in claim 1, wherein the housing further comprises a volume that is between 0.5 and 2 cm³.

14. The heart stimulator as claimed in claim 1, wherein the heart stimulator further comprises a weight that is less than 5 gr.

15. The heart stimulator as claimed in claim 1, further comprising a thread mount on the housing that temporarily actively fixes the heart stimulator.
